(19) United States
(12) Patent Application Publication
(10) Pub. No.: US 2015/0147780 A1
(43) Pub. Date: May 28, 2015

(54) METHOD FOR HARVESTING ORGANIC COMPOUNDS FROM GENETICALLY MODIFIED ORGANISMS

(71) Applicant: World Biotechnology LLC, North Brunswick, NJ (US)

(72) Inventors: Eugene Dinescu, North Brunswick, NJ (US); Vincent Dinescu, North Brunswick, NJ (US)

(21) Appl. No.: 14/551,816

(22) Filed: Nov. 24, 2014

Related U.S. Application Data
(60) Provisional application No. 61/907,857, filed on Nov. 22, 2013.
(60) Provisional application No. 61/929,128, filed on Jan. 20, 2014.

(51) Int. Cl. C12P 23/00; C12N 9/24; C12N 9/08; C12N 9/02; C12N 9/16; C12N 9/14; C12N 9/90; C12N 9/12; C12N 9/00; C12N 9/88; C12N 9/48; C12N 9/10; C12P 7/02; C07K 14/575; C07K 14/555; C07K 14/54; C07K 16/00; C07K 14/00; C12N 9/80

(52) U.S. Cl. CPC C12P 23/00; C12N 9/80; C12N 9/2402; C12N 9/0065; C12N 9/0004; C12N 9/16; C12N 9/14; C12N 9/90; C12N 9/12; C12N 9/93; C12N 9/88; C12N 9/1241; C12N 9/48; C12N 9/10; C12P 7/02; C07K 14/575; C07K 14/555; C07K 14/54; C07K 16/00; C07K 14/00
USPC 435/67; 435/228; 435/201; 435/192; 435/189; 435/196; 435/195; 435/233; 435/194; 435/232; 435/193; 435/132; 435/69.4; 435/69.51; 435/69.52; 435/69.6; 435/69.1

(57) ABSTRACT

Disclosed herein are embodiments for a novel method of producing an organic compound, including harvesting at least one organic compound from an organism or cell line genetically engineered with a gene for at least one proton-pump protein.

This application claims the benefit of U.S. Provisional Application No. 61/907,857, filed Nov. 22, 2013 and U.S. Provisional Application No. 61/929,128, filed Jan. 20, 2014. The contents of these applications are hereby incorporated by reference in their entireties.

### FIELD OF INVENTION

The present disclosure is directed to harvesting organic compounds, including biologics and biofuels, from genetically modified organisms.

### BACKGROUND

Genetically modified organisms offer opportunities to create and harvest a variety of organic compounds. Among these organic compounds are biologics and biofuel. To increase production efficiency of a biologic or biofuel, either the quantity of input must be reduced, the rate of production must be increased or the quality of the product must be improved. These areas can be addressed by boosting efficiency by providing an energy source from broad spectrum light.

To create cellular products, such as biologics and bioethanol, organisms require an energy source in the form of adenosine triphosphate (ATP). Generally, ATP is produced when a proton gradient is created across a cellular membrane. This proton motive force drives the production of ATP as protons move down the gradient through an ATP synthase. While this proton gradient can ultimately generate energy in the form of ATP, there is also an ATP energy cost to first pump protons across the membrane and against the gradient. There exists a need in the art for a system to minimize the energy costs when creating this proton gradient and to increase ATP output while avoiding cellular pathways that have negative feedback loops.

Disclosed herein is a system and composition which utilizes genetically engineered organisms and cell lines to use proton-pump proteins to increase creation of ATP to improve the organism's and cell line's productivity. Certain proton-pumps, such as bacteriorhodopsin, are light-driven and can create a proton gradient utilizing the light absorbed by the sun or other light source as an energy source. By relying on sunlight for energy, the organism or cell line does not require as much energy from other energy sources, such as glucose, glycogen, trehalose, NADH and FADH₂, to produce ATP. As a result, the organisms can be more efficient in their energy usage and production.

### OBJECTS AND SUMMARY OF THE INVENTION

It is an object of certain embodiments of the present invention to provide a method of harvesting an organic compound from an organism or cell line genetically engineered with a gene for at least one proton-pump protein.

It is an object of certain embodiments of the present invention to provide a method to genetically engineer bacteria, fungi, plants, fish, birds, mammals and cell lines to include at least one light-driven, proton-pump protein.

It is an object of certain embodiments of the present invention to provide a method to genetically engineer a proton-pump protein to include a mitochondrial targeting sequence so that the proton-pump protein is inserted into the inner mitochondrial membrane.

It is an object of certain embodiments of the present invention to provide a method to increase the titer of a biologic or biofuel.

It is an object of certain embodiments of the present invention to provide a method to increase production of a biologic where the biologic may be allergenics, antibodies, blood products or derivatives thereof, enzymes, growth factors, hormones, immunomodulators, interferons, interleukins, polypeptides, proteins, serums, tissues, toxins or vaccines.

It is an object of certain embodiments of the present invention to provide a method to increase production of a biofuel where the biofuel may be biodiesel, biogas, butanol, ethanol or methanol.

It is an object of certain embodiments of the present invention to provide a method to increase the production of ATP and organic compounds without increasing production input.

It is an object of certain embodiments of the present invention to provide a method to increase ATP-dependent cellular functions.

### BRIEF DESCRIPTION OF THE FIGURES

FIG. 1 is a generalized flow chart of the method demonstrating the process for selecting an organism or cell line and integrating selected genes to be used in harvesting organic compounds.

FIG. 2 is a specified flow chart for the genetic engineering of an already selected organism and proton-pump protein to be used in harvesting organic compounds.

### DETAILED DESCRIPTION

The present invention is directed to a method of harvesting an organic compound from an organism or cell line genetically engineered with a gene for at least one proton-pump protein and a composition with an organism or cell line genetically engineered with a gene for at least one proton-pump protein. The process for harvesting an organic product may include genetically engineering an organism or cell line to include at least one proton-pump protein, growing the organism or cell line and then harvesting a desired organic compound. In particular embodiments, the organic compound is a biologic or a biofuel.

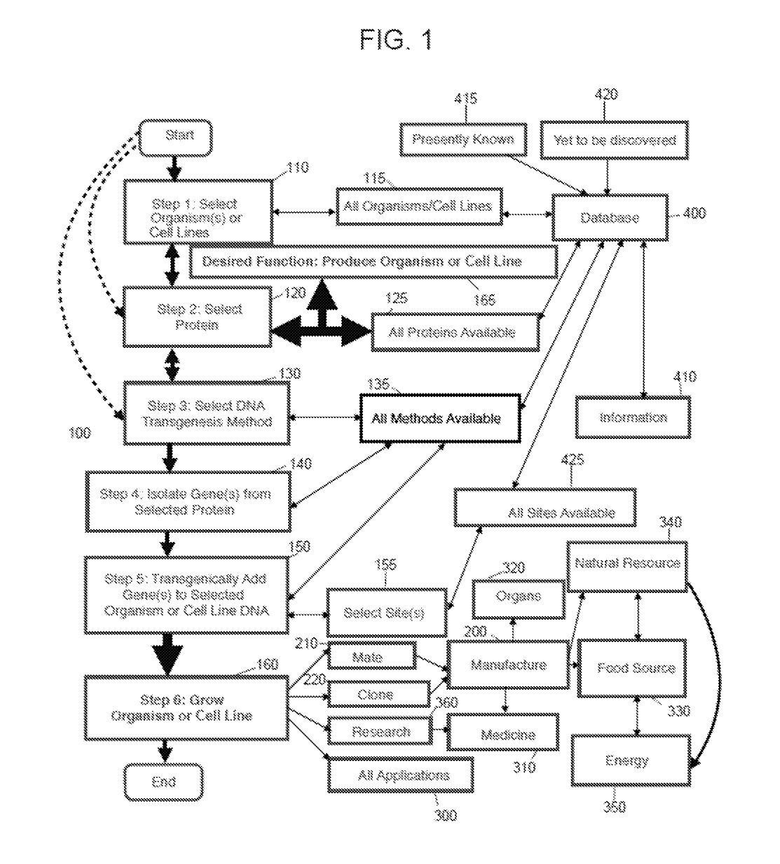

FIG. 1 shows a flow chart demonstrating the disclosed method. In some embodiments, the method may include a genetic engineering function process 100, organism or cell line manufacturing 200, a plurality of additional applications 300 and databasing 400. The genetic engineering function process 100 may include a selection of at least one organism or cell line 110, a protein selection of at least one protein 120, a transgenic methodology 130, a gene isolation 140, a gene addition 150 and organism or cell line growth 160.

The organism or cell line growth 160 may result in a genetically engineered organism or cell line 165. The genetically engineered organism or cell line may then develop as usual. The genetically engineered organism or cell line may then express/produce the proton-pump protein in cells and produce a proton gradient and ultimately ATP from light.

The organism selection 110 may include selecting from any known organism or cell lines 115. Selection may include all presently known organisms or cell lines 415 or all yet to be discovered or created organisms or cell lines 420.

The protein selection 120 may include all available proteins 125 including all proton-pump proteins. In certain embodiments, the proton-pump protein may include those proteins with conformational changes upon light absorption and pump protons through a membrane. The proton-pump protein may allow the organism to produce energy via ATP synthase. The proton-pump protein may be expressed in mitochondria and this may allow ATP production via light absorption. In certain embodiments, the proton-pump protein may be a combination of two or more proteins that can be utilized to enhance or facilitate the light absorption function.

The transgenic methodology 130 may include all methods known to those of skill in the art 135 to perform transgenesis.

The gene isolation 140 may include isolating a gene that codes for a proton-pump protein by any known method to those of skill in the art. The gene isolation 140 may include using restriction enzymes and gel electrophoresis. In certain embodiments, polymerase chain reaction (PCR) may be used to amplify the gene segment. In an alternate embodiment, the gene sequence for the proton-pump protein may be found in known DNA databases 400.

Gene addition 150 may include transgenically adding a gene to the genome of the selected organism or cell line 110 by all methods available 135. In certain embodiments, the gene addition 150 occurs at a selected site in the organism's or cell line's genome 155 which may include all known sites available 425. In other embodiments, the method may add the gene to the germ line of the selected organism 110, by any known method 150 to those of skill in the art, including, but not limited to, by injecting the foreign DNA into the nucleus of a fertilized ovum.

It should be understood that the organism or cell line selection 110, the protein selection 120 and the transgenic methodology 130 may be performed in any combination and does not need to follow any particular order.

The genetic engineered organism or cell line manufacturing 200 may include breeding 210, cloning 220 as well as other known methods to those of skill in the art. The genetically engineered organism or cell line manufacturing 200 may also engineer desired characteristics through any known method to those of skill in the art, such as cross-breeding or the like. For example, if enzyme A, B, C, D, E, and F are needed for complete amino acid synthesis in organism AA one can transgenically create a total of 6 organisms that all express a single missing enzyme. One organism will express A, another B, another C, another D, another E, and another F. Once this is accomplished these organisms can be crossbreed until production of a hybrid that expresses each enzyme is attained.

The plurality of additional applications 300 may include research 360, medicine 310, stem cell research and host organism production 320, a production of food source 330 or natural resource 340 including energy production 350.

The databasing 400 may include producing a database of all possible types of combinations and information 410 for producing the desired organism or cell line 165.

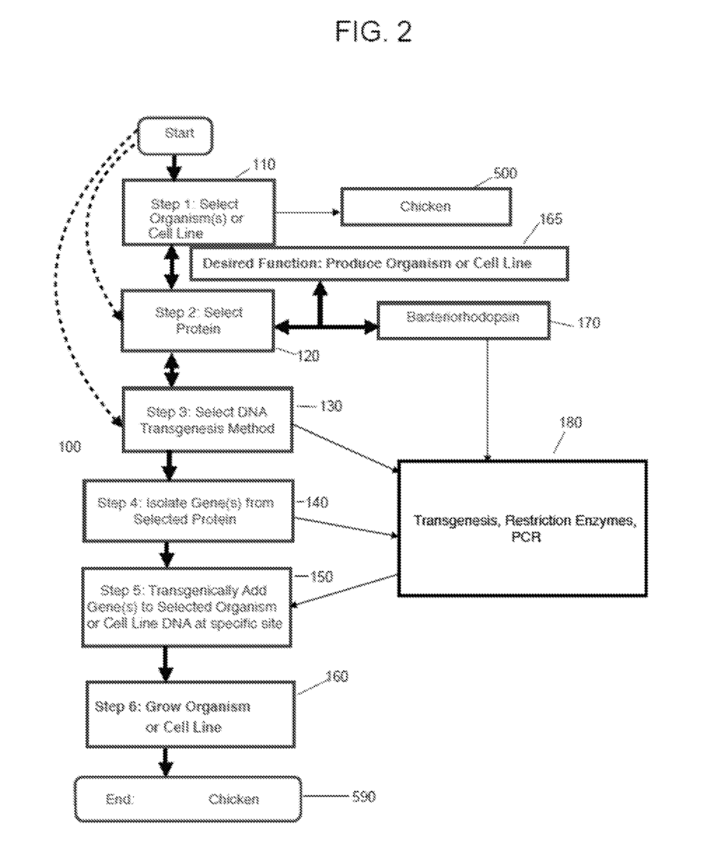

Referring to FIG. 2, the genetically engineering function process 100 may include creating a transgenic chicken 590. The following example is offered to be illustrative but no way limiting in describing the genetically engineering function process 100. To begin, a chicken is selected 500 as the known organism 110. The protein selection 120 may include selecting bacteriorhodopsin 170, an integral membrane protein that has Vitamin A attached. Bacteriorhodopsin effectively absorbs green light (wavelength 500-650 nm, with absorption maximum obtained at 568 nm) and is a protein used by archaea organisms. The bacteriorhodopsin is a proton-pump which changes conformation once it absorbs light and pumps protons through membrane. The bacteriorhodopsin may be expressed in mitochondria and may allow ATP production via light absorption as chemical energy. The bacteriorhodopsin gene can be isolated and amplified using techniques known to those of skill in the art, including restriction enzymes and gel electrophoresis to isolate the gene and PCR can be used to amplify gene segment 180. However, as aforementioned, the gene sequence for bacteriorhodopsin can easily and readily be found in DNA databases. The DNA trangenesis method 130 may be selected to best optimize integration based on the selected chicken 500 model and selected bacteriorhodopsin protein 170 known to those of skill in the art. The gene will be isolated 140 and then transgenically added 150 through techniques known to those of skill in the art, including adding the gene to the chicken's germ line by injecting the foreign DNA into the nucleus of a fertilized ovum. After transgenesis, the genetically engineered chicken may grow 160 as its non-genetically engineered counterpart.

Once this is accomplished the transgenic chicken 590 can then develop as usual. The chicken will now express/produce bacteriorhodopsin in cells. The transgenic chicken 590 will be able to produce a proton gradient from light. The transgenic chicken 590 will require less feed than a non-transgenic chicken 500.

While FIGS. 1 and 2 show a flow chart demonstrating versions of the method, it should be understood the method may be performed in any combination and does not necessarily need to be in any order.

### Direct-Light Technology

The current method may utilize proton-pump protein to help create a proton gradient across a cell membrane, including a light-driven, proton-pump. When a light-driven, proton-pump absorbs light, the protein generally forms a channel through a cellular membrane and undergoes a series of conformational changes in response to the absorbed light. The conformational changes allow protons to pass to and from different amino acid groups along the protein channel and through a cellular membrane. Moving the protons through the proton-pumps allows a proton gradient to be formed with enough proton motive force to drive an ATP synthase to make ATP. In some embodiments of the current invention, the light-driven, proton-pump protein may be archaerhodopsin, bacteriorhodopsin, opsin, proteorhodopsin, rhodopsin, xanthorhodopsin, homologs or combinations thereof. In certain embodiments, the proton-pump protein may include bacteriorhodopsin, an integral membrane protein that has Vitamin A attached and ability to absorb light.

Proton-pump proteins are able to operate after absorbing light from a wide range of the light spectrum. For instance, bacteriorhodpsin may generally best absorb light between 500 nm to 650 nm which corresponds to green light in the visible spectrum. Likewise, proteorhdopsin may maximally absorb light around 525 nm (green light) as well as around 490 nm (blue light) and rhodopsin will generally absorb light from around 490 nm to around 510 nm. While these are optimal ranges, proton-pump proteins are able to absorb light well above and below these peaks. In other embodiments, the proton-pump protein may absorb light between about 100 nm and about 1 μm, between about 300 nm and about 750 nm, between about 450 nm and about 650 nm, between about 450 nm and about 550 nm and between about 550 nm and about 600 nm.

In some embodiments of the present invention, the method is able to produce a variety of organic compounds, including biologics or biofuels. Biologics are generally considered to be large, complex molecules which are often produced by living cells and organisms naturally or through genetic engineering. Biologics may be used for a variety of uses, including disease treatments, diagnostics and prevention of a variety of health conditions. Unlike drugs, which can be produced on a large scale by chemical means, it remains very difficult to reproduce biologics outside of a living organism or cell line. To help solve this problem, embodiments of the current invention will utilize living organisms and cell lines to increase production of biologics. In some embodiments, the biologic may be allergenics, antibodies, blood products or derivatives thereof, enzymes, growth factors, hormones, immunomodulators, interferons, interleukins, polypeptides, proteins, serums, tissues, toxins and vaccines.

In particular embodiments, where the biologic is an antibody, the antibody may be, but not limited to, antitoxins, IgA, IgD, IgE, IgG, IgM antibodies or combinations thereof and may be either a monoclonal, polyclonal or bispecific antibody. In other embodiments, where the biologic is a blood product, the blood product may be, but not limited to, red blood cells, blood plasma, white blood cells, platelets, derivatives thereof or combinations thereof.

In some embodiments, where the biologic is an enzyme, the enzyme may be, but not limited to, an amidase, amylase, catalase, cellulase, dehydrogenase, endonuclease, hemicellulase, hydrolase, isomerase, kinase, ligase, lipase, lyase, lysozyme, pectinase, peroxidase, phosphatese, polymerase, protease, oxidase, oxidoreductase, reductase, transferase or combinations thereof.

In other embodiments, where the biologic is a hormone, the hormone may be, but is not limited to, adiponectin, adrenocorticotropic hormone, androgen, angiotensinogen, antidiuretic hormone, amylin, atrial-natriuretic peptide, brain natriuretic peptide, cacitonin, cholecystokinin, cortisol, corticotrophin-releasing hormone, cortistatin, enkephalin, endothelin, epinephrine, estrogen, erythropoietin, follicle-stimulating hormone, galanin, gastric inhibitory polypeptide, gastrin, ghrelin, glucagon, glucagon-like peptide-1, glucocorticoid, gonadotropin-releasing hormone, growth hormone, growth hormone-releasing hormone, hepcidin, human chorionic gonadotropin, human placental lactogen, humoral factors, inhibin, insulin, insulin-like growth factor, leptin, leukotriene, lipotropin, luteinizing hormone, melatonin, melanocyte stimulating hormone, mineralocorticoid, motilin, orexin, oxytocin, pancreatic polypeptide, parathyroid, pituitary adenlate cyclase-activating peptide, progesterone, prolactin, prolactin releasing hormone, prostacyclin, prostaglandins, relaxin, renin, secosteroid, secretin, somatostatin, testosterone, thrombopoietin, thromboxane, thyroid-stimulating hormone, thyrotropin-releasing hormone, thyroxine, triiodothyronine, vasoactive intestinal peptide or combinations thereof.

In addition, in certain embodiments, a genetically engineered organism or cell line may have additional genes for certain hormones included to increase hormone production within the organism or cell line. This may provide an additional production advantage to the genetically engineered organism or cell line. Additionally, the genetically engineered organism or cell line may be injected with peptide hormones responsible for hormone production.

In still other embodiments, where the biologic is an interferon, the interferon may be, but not limited to, interferon type I, interferon type II, interferon type III or combinations thereof. In some embodiments, where the biologic is an interleukin, the interleukin may be, but not limited to, interleukin-1, interleukin-2, interleukin-3, interleukin-4, interleukin-5, interleukin-6, interleukin-7, interleukin-8, interleukin-9, interleukin-10, interleukin-11, interleukin-12, interleukin-13, interleukin-14, interleukin-15, interleukin-16, interleukin-17, interleukin-18, interleukin-19, interleukin-20, interleukin-21, interleukin-22, interleukin-23, interleukin-24, interleukin-25, interleukin-26, interleukin-27, interleukin-28, interleukin-29, interleukin-30, interleukin-31, interleukin-32, interleukin-33, interleukin-34, interleukin-35, interleukin-36, interleukin-37 or combinations thereof.

In particular embodiments, where the biologic is a vaccine, the vaccine may be, but not limited to a whole-cell vaccine, DNA vaccine, RNA vaccine, protein-based vaccine, peptide-based vaccine, attenuated organism vaccine, attenuated virus or combinations thereof. For this particular embodiment, the vaccine may, but is not limited to, providing immunity from African swine fever, anthrax, bubonic plague, cervical cancer, chicken pox, Coxsackie, dengue fever, diphtheria, Ebola, echovirus, encephalitis, gastroenteritis, hepatitis, herpes, human immunodeficiency disease (HIV-1 or HIV-2), influenza, lower respiratory tract infection, Lyme disease, Marburg, measles, monkeypox, mumps, Norwalk virus infection, papillomavirus, parainfluenza, parvovirus, pertussis, picorna virus infection, pneumonia, pneumonic plague, polio, rabies, rotavirus infection, rubella, shingles, smallpox, swine flu, tetanus, tuberculosis, typhoids or yellow fever.

In addition, some embodiments of the present invention may be used to treat conditions including, but not limited to, ankylosing spondylitis, autoimmune diseases, cancer, Crohn's disease, diabetes, gout, indeterminate colitis, inflammatory bowel disease, psoriasis, psoriatic arthritis, rheumatoid arthritis, ulcerative colitis, uveitis and viral infection.

Embodiments of the current invention may also be used to increase production of biofuels. Biofuels are considered to be energy sources derived from organic materials. Biofuels are most widely used in liquid form which may be more easily integrated into currently used systems; ethanol as a biofuel is particularly known for this feature. Biofuels also have the feature of being transportable sources of energy. The use of biofuels may be preferable to other renewable energy sources, such as wind, solar, hydrothermal and tidal flows, which would require additional input to make these other energy sources compatible with presently used infrastructure. Biofuels may be produced through fermentation of organic material or through extraction of lipids, vegetable oils and animal fats.

To increase the efficiency of production of ethanol, some embodiments of the current invention allows genetically engineered organisms and cell lines to increase the amount of ethanol and other biofuels produced during manufacturing. Embodiments of the current invention may increase the proton gradient and available ATP which will then provide the engineered organism or cell line with an abundance of energy to increase production of a biofuel. In some embodiments, where the organic compound is a biofuel, the biofuel may be, but is not limited, biodiesel, biogas, butanol, ethanol or methanol. In particular embodiments, the biofuel may be ethanol. In certain other embodiments, the method may be used for other biomolecules including, but not limited to, spider silk, cartilage, exoskeleton structures and the like.

Typically, proton-pumping proteins are located in a cellular membrane. In order to effectively create a proton gradient, protons need to be kept separated-usually by the cellular membrane. In prokaryotic cells (i.e., cells without membrane-bound organelles, including mitochondria), proton-pump proteins may be located in plasma among other membranes. In some embodiments, the proton-pump protein may be integrated into the inner mitochondrial membrane. In particular embodiments, the proton-pump may be integrated into two or more cellular membranes. In some embodiments, the proton-pump may be integrated into the same cellular membranes and, in other embodiments, the cellular membrane may be integrated into different cellular membranes.

One feature of the current invention is the creation and maintenance of a proton gradient. In order to create this gradient, the proton-pump protein should be integrated into at least one cellular membrane and correctly oriented in relation to the native ATP synthase. To ensure a protein is properly placed into a membrane, a protein gene may have a membrane targeting sequence. After a protein is translated, the targeting sequences enable the cellular machinery to transport the protein to its proper location. In other embodiments, the gene for the proton-pump protein may be genetically engineered to include at least one membrane targeting sequence. In particular embodiments, the targeting sequence may be a mitochondrial targeting sequence or other specific membrane targeting sequence. Where some of the embodiments use a mitochondrial targeting sequence, the mitochondrial targeting sequence may be from the ATP, COX IV or RIP1 genes or homologs thereof.

### Method Systems and Technologies

In some embodiments, the method may include genetically engineered genes. In particular embodiments, the gene of the proton-pump protein is genetically engineered to include a selectable marker. To ensure a gene is properly integrated into the genome of the targeted organism or cell line, a selectable marker may be used. A selectable marker is generally a gene or part of a gene which is also inserted with a gene of interest. The selectable marker provides an additional, non-native characteristic to the organism or cell line to distinguish the organisms or cell lines with the gene of interest and selectable marker from the organisms and cell lines without it.

In some embodiments, the selectable marker may be a drug resistance marker, a multidrug resistance marker, a metabolic survival marker, a color marker, a fluorescent marker or a combination thereof. In particular embodiments, the selectable marker may be dihydrofolate reductase gene, a guanosine phosphoribosyl transferase (GPT) gene, histidinol resistance gene, hygromycin resistance gene, β-galactosidase gene, green fluorescent protein gene, red fluorescent protein gene, blue fluorescent protein gene, yellow fluorescent protein gene, dsRed fluorescent protein gene, zeomycin resistance gene, zeocin resistance gene, puromycin resistance gene, Blacsticidin S resistance gene, spectinomycin resistance gene, streptomycin resistance gene and a neomycin resistance gene.

The invention may require integration of proton-pump genes or other genetically engineered genes that are not native to a selected organism's or cell line's genome. To integrate the proton-pump gene into a genome, a variety of techniques, known to those skilled in the art, may be used. Some embodiments may use genetically engineering an organism or cell with techniques including, but not limited to, breeding, calcium phosphate precipitation, chemical poration, cloning, conjugation, DEAE-dextran mediated transfection, electroporation, homologous recombination, non-homologous recombination, laser irradiation, lipofection, natural transformation, magnetofection, microinjection, particle bombardment, PEG poration, protoplast fusion, retroviral delivery, silicon fiber delivery, sonoporation, transfection, transformation or transduction. In particular embodiments where genetic engineering is through transduction, a lentivirus may be used.

Some organisms, such as with Saccharomyces cerevisiae or Schizosaccharomyces pombe, are unable to produce retinal which is a required co-factor for the proper functioning of bacteriorhodopsin. In some embodiments, depending on the organism or cell line used, the method further comprises growing the organism with co-factor retinal. As an alternative in other embodiments, an organism or cell line which is unable to produce retinal may be genetically engineered to include genes for crtE, crtYB, crtI and Bcmo1 or homologs thereof which may be used as a set of genes to make β-carotene which may be converted to retinal.

The method may be used with a variety of organisms. The organism may be selected from the bacterial, eukaryotic and archaic kingdoms, which may include, but not limited to, bacteria, fungi, plants, fish, birds and mammals.

In some embodiments, the organism may be a bacteria. The bacteria may be, but not limited to, Agrobacterium tumefaciens, Bacillus brevis, Bacillus licheniformis, Bacillus subtilis, Escherichia coli, Paenibacillus, Penicillium griseofulvum, Pseudomonas fluorescens, Ralstonia eutropha, Streptomyces aureofaciens, Streptomyces fradiae, Streptomyces lincolnensis, Streptomyces rimosus or Streptomyces venezuelae.

In other embodiments, the organism may be a fungus. In particular cases, the fungus may be a yeast. The fungus may be, but not limited to, Acremonium chrysogenum, Aspergillus awamori, Aspergillus nidulans, Aspergillus niger, Aspergillus rugulosus, Chrysosporium lucknowense, Hansenula polymorpha, Pichia pastoris, Saccharomyces cerevisiae or Schizosaccharomyces pombe.

In other embodiments, the organism may be an archaea. The archaea may be, but not limited to, Halobacterium salinarum or Pyrolobus fumarii.

In further embodiments, the organism may be a plant. The plant may be, but not limited to, alfalfa, algae, Arabidopsis thaliana, banana, bean, beet, Camelina sativa, canola, carrot, corn, legumes, palm, potato, rapeseed, rice, safflower, soybean, spinach, strawberry, sugarcane, sunflower, tobacco, tomato, turnip or wheat.

In particular embodiments, the plant may be an algae. The algae may be, but not limited to, Ahnfeltia, Alaria esculenta, Ankistrodesmus, Ascophyllum nodosum, Betaphycus gelatinum, Botryococcus braunii, Callophyllis variegate, Caulerpa, Chlorella protothecoides, Chlorella vulgaris, Chlamydomonas reinhardtii, Chondrus crispus, Cladosiphon okamuranus, Crypthecodinium cohnii, Dunaliella bardowil, Dunaliella salina, Dunaliella tertiolecta, Durvillaea, Ecklonia, Eucheuma, Gelidiella acerosa, Gelidium, Gracilaria, Haematococcus pluvialis, Hantzschia, Hizikia fusiformis, Isochrysis galbana, Kappaphycus, Laminaria, Lessonia, Macrocystis pyrifera, Mastocarpus stellatus, Monostroma, Mazzaella, Nannochloris, Nannochloropsis, Neochloris oleoabundans, Nitzschia, Palmaria palmate, Phaeodactylum tricornutum, Phymatolithon, Pleurochrysis carterae, Porphyra, Porphyridium, Sarcothalia, Sargassum, Scenedesmus, Schiochytrium, Stichococcus, Tetraselmis suecica, Thalassiosira pseudonana, Ulva or Undaria. In particular embodiments, the algae may be genetically engineered to have a higher lipid content in comparison to non-genetically modified algae.

In some embodiments, the organism may be a fish. The fish may be, but not limited to, carp, catfish, goldfish, loach, medaka, salmon, tilapia, trout or zebra fish.

In some embodiments, the organism may be a bird. The bird may be, but not limited to, a blackbird, canary, chicken, cockatoo, crow, duck, eagle, emu, falcon, finch, goose, hawk, jay bird, kiwi, macaw, mynah, ostrich, parakeet, parrot, partridge, pigeon, pheasant, quail, rhea, sparrow, toucan, turkey or warbler.

In other embodiments, the organism may be a mammal. The mammal may be, but not limited to, a bison, buffalo, bull, camel, cow, donkey, goat, horse, llama, mouse, non-human primate, oxen, pig, rabbit, rat or sheep.

In some embodiments, the method may utilize a cell line. In particular embodiments, the cell line may be a suspension cell line. In other embodiments, the cell line may be, but not limited to, 3T3, A549, Be2C, Caco2, CHO, Cos7, GT293, HEK 293, HepG2, HL60, HT1080, hybridoma, IMR90, Jurkat, K562, LnCap, MCF7, myeloma, N50, Namalwa, PC12, PER.C6, primary fibroblast, SKBR3, SW480, THP1, U 266B1, U937, WEHI 231 and YAC 1. In still other embodiments, the cell line is A549, CHO, HeLa, HEK 293, Jurkat or 3T3.

### Functions of the Method

The method may be used to help increase the productivity of a variety of cellular functions. Increasing these cellular functions may provide a greater yield of organic products, including for biologics and biofuels. In some embodiments, the method may be used to increase ATP production. As previously mentioned, ATP is produced when a proton travels through an ATP synthase which generates sufficient energy to bind a phosphate group to adenosine diphosphate creating ATP. By incorporating a greater number of proton-pumping proteins, more protons will be available to generate ATP. Thus, some embodiments may increase the proton gradient. Also, other embodiments may also increase ATP synthase activity as well. In some embodiments, high titers of organic compounds, including biologics and biofuels, may be produced from the increase of available energy.

In some embodiments, ATP-dependent cellular functions may also increase. Many cellular functions required energy in the form of ATP in order to occur. These cellular functions include metabolic reactions, macromolecule syntheses (i.e., DNA, RNA, proteins, carbohydrates, amino acids, lipids, fatty acids, ethanol etc.), signaling, fermentation, cell structure, cell movement, mitosis, meiosis, among many others. Also, by providing a possible alternative source of ATP, in other embodiments, the method may help to increase cellular energy conservation.

The possible increased amount of ATP may help increase protein folding. For example, when a protein is targeted to the mitochondrial membrane (such as a proton-pump protein with a mitochondrial targeting sequence), the protein must be unfolded from its native state in order to be imported into the mitochondria. Another example is when a protein is overexpressed, it may misfold and form an aggregate or it may be degraded. For these proteins to be correctly refolded, the process requires energy in the form of ATP along with the energy needs of chaperone, transport and other necessary proteins. In some embodiments, the method may increase the supply of ATP to increase protein folding.

In some embodiments, method may also help to increase production of various cellular products including, but not limited to, fatty acids, amino acids and ethanol. Fatty acids are important sources for energy and cellular structures as well as being utilized for biofuel production. However, fatty acids require ATP in their synthesis. Fatty acid synthesis occurs through the Type-I and Type-II fatty acid synthases and is encoded by the FASN gene and its homologs thereof. In some embodiments, organisms or cell lines may be further genetically engineered to also include the FAS1, FAS2, FASN genes, homologs or combinations thereof. The addition of the FAS1, FAS2, FASN genes, homologs or combinations thereof may help to increase fatty acid synthesis in some embodiments. Likewise, an increase in fatty acid production may increase the amount of animal fat in genetically engineered organisms which may increase the amount of biofuel and biodiesel produced.

Amino acids are essential for protein synthesis which also requires ATP energy input. Production of amino acids varies greatly depending on the organism. In some organisms, particular amino acids cannot be synthesized by the organism, as seen with humans. In some embodiments, organisms or cell lines may be further genetically engineered to also include the gene for an amino acid producing enzyme. In particular embodiments, the amino acid producing enzyme may be for the production of an amino acid including, but not limited, alanine, arginine, aspartate, asparagine, cysteine, glutamate, glutamine, glycine, histidine, isoleucine, leucine, lysine, methionine, phenylalanine, proline, serine, threonine, tyrosine, tryptophan or valine. In particular embodiments, the gene for the enzyme for amino acid synthesis may before the synthesis of lysine from aspartate.

In certain embodiments, the increased production of hormones, amino acids and fatty acids of the genetically modified organisms or cell line may help the genetically modified organism or cell lines to decrease dietary intake, grow faster than their non-genetically modified counterparts and be mass-produced for less than the current cost of production.

During normal respiration, organisms are able to use oxygen to convert sugars and carbohydrates into energy in the form of ATP. During fermentation, however, oxygen is not available and organisms (generally yeast and some bacteria) utilize the alternative fermentation process. The result of this process is continual production of ATP with ethanol being created as a byproduct. In some embodiments, the method may increase ethanol production by providing a continuing proton gradient.

In embodiments of the invention, the production of a proton gradient outside of the normal cellular processes may increase the production or synthesis of fatty acids, amino acids and ethanol.

The method may also help organisms and cell lines counteract the stresses associated with ethanol production. When ethanol levels reach a critical level within the cell, the ethanol can begin to denature proteins and increase membrane fluidity. If a membrane becomes too fluid, the proton gradient may be lost and other energy sources, such as glucose, glycogen, trehalose, NADH and FADH₂, may be consumed at greater rates to preserve the proton gradient. Denatured proteins may also begin to aggregate preventing the protein from properly functioning or trigger other cellular mechanisms to degrade and recycle those proteins.

In some embodiments, the method may increase ethanol tolerance. Cellular defenses against ethanol stresses include the upregulation of fatty acid elongation factor Elo1. Elo1 increases the proportion of acyl chains in the membrane from 18:1 to 16:1 in the membrane which helps stabilize the membrane from increased fluidity. In addition, cells can increase the production of chaperone and heat shock proteins to help refold denatured proteins and prevent aggregation. These cellular defenses all require significant amounts of energy, usually from sources like glucose, glycogen, trehalose, NADH and FADH₂. The consumption of trehalose to produce ATP may be particularly significant as trehalose helps to preserve membrane integrity, protein stability and suppress protein aggregation.

Since in some embodiments, the increase number of proton-pump proteins may increase the supply of the ATP as an energy source which may help facilitate the cellular defenses against ethanol stresses. In other embodiments, the method may preserve the accumulation of glucose, glycogen, trehalose, NADH and FADH₂; enhance the remodeling of the membrane, upregulate fatty acid elongation factor Elo1 and increase the proportion of acyl chains from 18:1 to 16:1 within the cellular membrane.

For a cell to counteract the stresses of ethanol, many of the cell's defense mechanisms require energy input. In some embodiments, the method and the increase of ATP production may decrease the toxic effects of ethanol stress; decrease protein aggregation; decrease glucose, glycogen, trehalose, NADH and FADH₂ consumption; and decrease the loss of the proton gradient.

In other embodiments, the method may decrease the glycolytic negative feedback loop of an organism or cell line. Typically, during cellular respiration, high ATP levels in the cell will help prevent further ATP from being produced. If there is too much free ATP in a cell, the excess ATP will bind to phosphofructokinase, an enzyme used in glycolysis which produces ATP, and will prevent further ATP production. Since the method provides for an independent source to create the proton gradient, the typical glycolytic negative feedback loop may be bypassed.

In some embodiments, certain genes for growth of hair, feather, scale or other similar structures may be deactivated so that the organism has the maximal amount of surface area to absorb light.

As a result of the various possible effects of integrating proton-pump proteins into the cell, in particular embodiments, the method may provide for a higher quality and a higher activity of organic compounds, including biologics and biofuels.

In some embodiments, the method may also use photocatalysts. In some embodiments, photocatalysts may be used to help generate free radicals and may produce hydrogen fuel. In other embodiments, hydrogen powered cells may be produced. In particular embodiments, the method may also further include metal catalysts. These metal catalysts may include, but are not limited to, chromium, copper, iron, nickel, platinum, palladium, titanium dioxide or zirconium dioxide. The protons that are pumped by the proton-pump proteins can then interact and bind to the free electrons at the metal sites that will be enclosed in a matrix of metal oxide. The metal oxide can be formed by titanium dioxide and zirconium dioxide. In some embodiments, hydrogen fuel may be produced through the method.

In other embodiments, the method may be coupled with molecular machines. Molecular machines may include cellular components to accomplish various tasks in the cell. Examples of molecular machines include mechanical components such as joints, valves, gears, propellers, ratchets and others to form machines that can act as motors, tweezers, vehicles, assembly lines, controlled release systems, switches, transportation networks, among others. In particular embodiments, the method may be used in conjunction with the F₀F₁-ATP Synthase motor to produce ATP.

In other embodiments, the method may also include use of a light-gated ion channel. Light-gated ion channels are pores that can transport materials through the pore in response to light. In particular embodiments, light-gated ion channels, such as channelrhodopsin or nicotinic acetylcholine receptor, may be used to produce electrical signals from light absorption. In particular embodiments, such electrical signaling may be utilized in computers, cars, airplanes, buildings, and any other non-organic application that utilizes this form of electrical signaling.

In certain embodiments, the method may be used by placing the proton-pump protein into a capsule-like enclosure which and may be able to produce hydrogen gas.

In certain embodiments, a method may include creating a facility equipped maximize light exposure and absorption of the proton-pump protein and its energy production. In particular embodiments, facility may include maximizing absorption of the proton-pump by providing lighting that may be adjusted to optimize absorption for the selected proton-pump and genetically modified organism.

In some embodiments, a composition may be prepared to include a genetically modified organism according to any of the disclosure above. In other embodiments, the composition may include an organism or cell line genetically engineered with a gene for at least one proton-pump protein, the organism or cell line having an increased yield of a desired organic compound.

In still other embodiments, a composition an organism or cell line genetically engineered with a gene for at least one proton-pump protein with a membrane targeting sequence, the organism or cell line having an increased yield of a desired organic compound. In particular embodiments, the composition may include an organism or cell line genetically engineered with a gene for at least one proton-pump protein with a mitochondrial targeting sequence, the organism or cell line having an increased yield of a desired organic compound.

The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### EXAMPLES

### Example 1

### Prophetic—Genetically Engineering Escherichia coli with Proteorhodopsin

Escherichia coli (E. coli) is selected to be genetically engineered with proteorhodopsin. Proteorhodopsin (PR) is a homolog of bacteriorhodpsin which functions as a light-driven, proton-pump. PR expresses well in E. coli as the preferred proton-pump for this system. Homologous recombination is selected to transform the E. coli.

Genomic DNA is isolated from an organism which contains a native gene for PR. The PR gene may be isolated through restriction enzyme and gel electrophoresis techniques known to those of skill in the art. Amplification of the isolated PR gene is performed through PCR reactions optimize to produce the highest yield and quality available for the PR gene. Primers for the PCR reaction may be designed include target gene sequences for homologous recombination with the targeted gene. In addition, primers may also be designed to add sequences for membrane targeting, and specifically, to the inner membrane of E. coli, where the ATPase synthase is located. After amplification, the PCR product is purified with techniques such as gel purification and precipitation

Also, β-carotene production genes may be inserted into the E. coli genome through homologous recombination or other techniques. E. coli do not naturally produce retinal (which is necessary for PR functioning). Thus, genes for crtE, crtYB, crtI and Bcmo1 or homologs thereof may be added to the E. coli to produce β-carotene which may be converted to retinal via the Bcmo1 enzyme. Alternatively, the transformed E. coli may be grown with a retinal supplemented media. Likewise, other desired gene additions, like additional proton-pump or fatty acid synthesis genes may be transformed into E. coli using the same or similar techniques.

An appropriate plasmid construct is chosen to include a desired selectable marker, such as drug resistance or color marker. The PR or modified PR PCR product is inserted into the plasmid construct. The E. coli is grown to an appropriate concentration for the desired transformation technique.

The plasmid is introduced to the E. coli through techniques such as electroporation. After electroporation, the E. coli is plated to incubate at least overnight. If using a selection technique, such as drug resistance, the electroporated cells are plated with the appropriate selection compound. After incubating overnight, test colonies growing on the selection plate are further cultured. PCR and DNA sequencing is used to confirm insertion of the PR or modified PR gene in the test E. coli colonies. SDS-PAGE and western blotting using antibodies raised against PR will be used to confirm the expression of the PR protein,

### Example 2

### Prophetic—Genetically Engineering Yeast with Bacteriorhodopsin

Yeast strains, such as Saccharomyces cerevisiae or Schizosaccharomyces pombe, may be transformed through homologous recombination with bacteriorhodopsin (BR) through the techniques described in Example 1. In the selected yeast strain, additional sequences (such as the membrane targeting sequences or mitochondrial targeting sequence) as well as other desirable genes may also be transformed into the yeast genome. Like E. coli, some yeast strains do not produce β-carotene or retinal. Thus, the appropriate genes (i.e., crtE, crtYB, crtI and Bcmo1 or homologs thereof) should be genetically engineered into the yeast genome or the appropriate supplemental retinal should be added in the same manner as the BR gene.

### Example 3

### Prophetic—Genetically Engineering Mammalian Cell Lines with Bacteriorhodopsin

Cell lines, such as HEK293T and CHO, are selected to be transformed with BR through transduction with lentivirus. The selected cell lines are prepared to the appropriate concentration. Similarly to Example 1, the BR is isolated and amplified from isolated genomic DNA. The BR gene and selection marker are inserted into a transfer vector with long terminal repeats (LTRs) and the Psi-sequence of HIV-1. Additional desired genes may also be included in the transfer vector. The desired infection system is created with the selected transfer vector plasmid, packaging plasmid and heterologous envelop vector plasmid best optimized for the cell line to create viral particles.

The lentiviral virus is added to the selected cell line to infect the cells. Positively infected cells carrying the BR gene are selected for by incubating with media with the appropriate selection compound. After incubating, test cells lines in the selection method and continue culture of the positively selected colonies. The infection event produces a mixed population of cells expressing the BR gene at different levels based on where the viral genome has integrated in the host genome. To optimize expression, single cell clones are individually sorted and expanded. The single cell clones are then tested for expression and classified by their expression level of the desired gene such as BR. PCR and DNA sequencing is used to confirm insertion and proper sequence of the BR or modified BR gene into the transduced cells.

In the foregoing description, numerous details are set forth. It will be apparent, however, that the disclosure may be practiced without these specific details. Whereas many alterations and modifications of the disclosure will no doubt become apparent to a person of ordinary skill in the art after having read the foregoing description, it is to be understood that any particular embodiment shown and described by way of illustration is in no way intended to be considered limiting. Therefore, references to details of various embodiments are not intended to limit the scope of the claims, which in themselves recite only those features regarded as the disclosure.

1. A method of producing an organic compound comprising,
harvesting at least one organic compound from an organism or cell line genetically engineered with a gene for at least one proton-pump protein.

2. The method of claim 1, wherein the at least one proton-pump protein is a light-driven proton-pump protein.

3. The method of claim 1, wherein the at least one proton-pump protein is an archaerhodopsin, bacteriorhodopsin, opsin, proteorhodopsin, rhodopsin, xanthorhodopsin, homologs thereof or combinations thereof.

4. The method of claim 3, wherein the at least one proton-pump protein absorbs light between about 100 nm and about 1 μm.

5. The method of claim 4, wherein the at least one proton-pump protein absorbs light between about 300 nm and about 750 nm.

6. The method of claim 5, wherein the at least one proton-pump protein absorbs light between about 450 nm and about 650 nm.

7. The method of claim 6, wherein the at least one proton-pump protein absorbs light between about 450 nm and about 550 nm.

8. The method of claim 6, wherein the at least one proton-pump protein absorbs light between about 550 nm and about 600 nm.

9. The method of claim 1, wherein the at least one organic compound is a biologic or a biofuel

10. The method of claim 9, wherein the biologic is selected from the group consisting of allergenics, antibodies, blood products or derivative thereof, enzymes, growth factors, hormones, immunomodulators, interferons, interleukins, polypeptides, proteins, serum, tissues, toxins and vaccines.

11. The method of claim 10, wherein the antibody is selected from the group consisting of an antitoxin, IgA, IgD, IgE, IgG, IgM and combinations thereof.

12. The method of claim 11, wherein the antibody is a monoclonal, polyclonal or bispecific antibody.

13. The method of claim 10, wherein the blood product is selected from the group consisting of red blood cells, blood plasma, white blood cells, platelets, derivatives thereof and combinations thereof.

14. The method of claim 10, wherein the enzyme is selected from the group consisting of amidases, amylases, catalases, cellulases, dehydrogenases, endonucleases, hemicellulases, hydrolases, isomerases, kinases, ligases, lipases, lyases, lysozymes, pectinases, peroxidases, phosphateses, polymerases, proteases, oxidases, oxidoreductases, reductases, transferases and combination thereof.

15. The method of claim 10, wherein the hormone is selected from the group consisting of adiponectin, adrenocorticotropic hormone, androgen, angiotensinogen, antidiuretic hormone, amylin, atrial-natriuretic peptide, brain natriuretic peptide, cacitonin, cholecystokinin, cortisol, corticotrophin-releasing hormone, cortistatin, enkephalin, endothelin, epinephrine, estrogen, erythropoietin, follicle-stimulating hormone, galanin, gastric inhibitory polypeptide, gastrin, ghrelin, glucagon, glucagon-like peptide-1, glucocorticoid, gonadotropin-releasing hormone, growth hormone, growth hormone-releasing hormone, hepcidin, human chorionic gonadotropin, human placental lactogen, humoral factors, inhibin, insulin, insulin-like growth factor, leptin, leukotriene, lipotropin, luteinizing hormone, melatonin, melanocyte stimulating hormone, mineralocorticoid, motilin, orexin, oxytocin, pancreatic polypeptide, parathyroid, pituitary adenlate cyclase-activating peptide, progesterone, prolactin, prolactin releasing hormone, prostacyclin, prostaglandins, relaxin, renin, secosteroid, secretin, somatostatin, testosterone, thrombopoietin, thromboxane, thyroid-stimulating hormone (TSH), thyrotropin-releasing hormone, thyroxine, triiodothyronine, vasoactive intestinal peptide and combinations thereof.

16. The method of claim 10, wherein the interferon is selected from the group consisting of interferon type I, interferon type II, interferon type III and combinations thereof.

17. The method of claim 10, wherein the interleukins is selected from the group consisting of interleukin-1, interleukin-2, interleukin-3, interleukin-4, interleukin-5, interleukin-6, interleukin-7, interleukin-8, interleukin-9, interleukin-10, interleukin-11, interleukin-12, interleukin-13, interleukin-14, interleukin-15, interleukin-16, interleukin-17, interleukin-18, interleukin-19, interleukin-20, interleukin-21, interleukin-22, interleukin-23, interleukin-24, interleukin-25, interleukin-26, interleukin-27, interleukin-28, interleukin-29, interleukin-30, interleukin-31, interleukin-32, interleukin-33, interleukin-34, interleukin-35, interleukin-36, interleukin-37 and combinations thereof.

18. The method of claim 10, wherein the vaccine is selected from the group consisting of whole-cell vaccine, DNA vaccine, RNA vaccine, protein-based vaccine, peptide-based vaccine, attenuated organism vaccine, attenuated virus and combinations thereof.

19. The method of claim 18, wherein the vaccine provides immunity from a disease selected from the group consisting of acquired immune deficiency syndrome, African swine fever, anthrax, bubonic plague, cervical cancer, chicken pox, Coxsackie, dengue fever, diphtheria, Ebola virus, echovirus, encephalitis, gastroenteritis, hepatitis, herpes, human immunodeficiency disease (HIV-1 or HIV-2), influenza, lower respiratory tract infection, Lyme disease, Marburg, measles, monkeypox, mumps, Norwalk virus infection, papillomavirus, parainfluenza, parvovirus, pertussis, picorna virus infection, pneumonia, pneumonic plague, polio, rabies, rotavirus infection, rubella, shingles, smallpox, swine flu, tetanus, tuberculosis, typhoids and yellow fever.

20. The method of claim 10, wherein the biologic is used to treat a condition selected from the group consisting of ankylosing spondylitis, autoimmune diseases, cancer, Crohn's disease, diabetes, gout, indeterminate colitis, inflammatory bowel disease, psoriasis, psoriatic arthritis, rheumatoid arthritis, ulcerative colitis, uveitis and viral infection.

21-104. (canceled)
